(19) United States
(12) Patent Application Publication
(10) Pub. No.: US 2025/0177000 A1
(43) Pub. Date: Jun. 5, 2025

(54) IMPLANT AND SUTURING DEVICES

(71) Applicant: HANGZHOU REJOIN MASTIN MEDICAL DEVICE CO., LTD., Hangzhou (CN)

(72) Inventors: Jiancheng HU, Hangzhou (CN); Bin LI, Hangzhou (CN); Shengjun XU, Hangzhou (CN)

(73) Assignee: HANGZHOU REJOIN MASTIN MEDICAL DEVICE CO., LTD., Hangzhou (CN)

(21) Appl. No.: 19/019,391

(22) Filed: Jan. 13, 2025

Related U.S. Application Data
(63) Continuation-in-part of application No. PCT/CN2023/111292, filed on Aug. 4, 2023.

(30) Foreign Application Priority Data
Aug. 11, 2022 (CN) 202210964132.0

(51) Int. Cl. A61B 17/34; A61B 17/00; A61B 17/04

(52) U.S. Cl. CPC A61B 17/3468; A61B 17/0491; A61B 2017/00367

(57) ABSTRACT

The embodiments of the present disclosure provide an implant and a suturing device. A first chamfer is provided between a front end surface of the implant and a circumferential sidewall of the implant. A second chamfer is provided between a rear end surface of the implant and the circumferential sidewall of the implant. The suturing device comprises a puncture needle and one or more implants. The one or more implants are located on the puncture needle. The puncture needle is provided with a channel extending in an axial direction. The one or more implants can be triggered from the suturing device through the channel. At least two limiting structures are provided on an inner wall of the channel. The at least two limiting structures are arranged along a circumferential direction; and the at least two limiting structures can at least block a second implant to be triggered, such that an applied force is increased when the second implant to be triggered is triggered out of the suturing device.

### CROSS-REFERENCE TO RELATED APPLICATIONS

This application is a continuation-in-part of the International Application No. PCT/CN2023/111292, filed on Aug. 4, 2023, which claims priority to Chinese Patent Application No. 202210964132.0, filed on Aug. 11, 2022, the entire contents of each of which are incorporated herein by reference.

### TECHNICAL FIELD

The present disclosure relates to the technical field of medical devices, and in particular to an implant and a suturing device.

### BACKGROUND

When suturing tissues with a suturing device, a puncture needle may encounter an obstacle during puncturing, causing the puncture needle to bend slightly, and an implant disposed on the puncture needle may squeeze the implant upon contact with a sidewall of the bent puncture needle to cause the implant to break.

Therefore, it is desirable to provide an implant and a suturing device to alleviate the problem of breakage of the implant caused by the existing implant being squeezed after the puncture needle is bent.

### SUMMARY

One or more embodiments of the present disclosure provide an implant. A first chamfer may be provided between a front end surface of the implant and a circumferential sidewall of the implant; and a second chamfer may be provided between a rear end surface of the implant and the circumferential sidewall of the implant.

One or more embodiments of the present disclosure provide a suturing device. The suturing device may comprise a puncture needle and one or more implants. A first chamfer may be provided between a front end surface of each of the one or more implants and a circumferential sidewall of the implant, and a second chamfer may be provided between a rear end surface of each of the one or more implants and the circumferential sidewall of the implant. The one or more implants may be disposed on the puncture needle. The puncture needle may be provided with a channel extending in an axial direction. The one or more implants are capable of being triggered from the suturing device through the channel. At least two limiting structures may be disposed on an inner wall of the channel and arranged along a circumferential direction. The at least two limiting structures are capable of at least blocking a second implant to be triggered, such that an applied force is increased when the second implant to be triggered is triggered from the suturing device.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, wherein:

FIG. 1 is a schematic diagram illustrating an implant according to some embodiments of the present disclosure;

FIG. 2a is a schematic diagram illustrating a suturing device according to some embodiments of the present disclosure; FIG. 2b is a schematic diagram illustrating a suturing device according to some embodiments of the present disclosure; FIG. 2c is a schematic diagram illustrating a top view of a suturing device according to some embodiments of the present disclosure;

FIG. 3a is a schematic diagram illustrating a partial enlargement of a position A in FIG. 2a; FIG. 3b is a schematic diagram illustrating a partial enlargement of a position C in FIG. 2b; FIG. 3c is a schematic diagram illustrating a partial enlargement of a position E in FIG. 2c;

FIG. 4 is a schematic diagram illustrating a cross-section of a puncture needle of a suturing device according to some embodiments of the present disclosure;

FIG. 5 is a schematic diagram illustrating a suturing device excluding an upper half housing according to some embodiments of the present disclosure;

FIG. 6 is a schematic diagram illustrating a front view of a suturing device according to some embodiments of the present disclosure;

FIG. 7 is a schematic diagram illustrating a cross-sectional view in an A-A direction in FIG. 6;

FIG. 8 is a schematic diagram illustrating a cross-sectional view in a B-B direction in FIG. 6;

FIG. 9 is a schematic diagram illustrating a gear slot of a suturing device according to some embodiments of the present disclosure;

FIG. 10 is a schematic diagram illustrating a movement path of a free end in the gear slot in FIG. 9;

FIG. 11 is a schematic diagram illustrating another gear slot of a suturing device according to some embodiments of the present disclosure; and

FIG. 12 is a schematic diagram illustrating a movement path of a free end in the gear slot in FIG. 11.

Reference signs: 100—puncture needle; 110—channel; 111—notch, 112—slot structure, 113—protrusion; 114—tip end; 120—limiting structure; 121—guide slope; 210—first implant; 220—second implant; 230—first portion; 240—second portion; 310—deformation clearance; 320—first chamfer; 330—second chamfer; 400—fixing portion; 510—push pin; 520—push button; 530—connection arm; 540—linkage portion; 550—spring; 610—depth limiting tube; 621—first connection arm; 622—pressing arm; 6221—gear pin; 623—second connection arm; 630—limiting slot; 640—first guide slot; 650—sliding abutting portion; 660—button; 710—first longitudinal rod; 720—first vertical rod; 730—second longitudinal rod; 740—second vertical rod; 750—limiting buckle; 760—free end; 810—first slot unit; 811—first starting end; 812—first limiting end; 813—second slot wall; 820—first retreat slot; 830—second slot unit; 831—second starting end; 832—second limiting end; 840—first transverse slot; 850—second transverse slot; 860—third transverse slot; 870—second retreat slot; 900—housing; 910—threaded member.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system”, “device”, “unit” and/or “module” used herein are a way to distinguish between different components, elements, parts, sections, or assemblies at different levels. However, the terms may be replaced by other expressions if other words accomplish the same purpose.

As shown in the present disclosure and in the claims, unless the context clearly suggests an exception, the words “one”, “a”, “an”, “one kind”, and/or “the” do not refer specifically to the singular, but may also include the plural. Generally, the terms “including” and “comprising” suggest only the inclusion of clearly identified steps and elements, however, the steps and elements that do not constitute an exclusive list, and the method or apparatus may also include other steps or elements.

An implant is an active or passive medical product that is implanted in a human body or other living organisms for long-term or temporary treatment or diagnosis purposes. The implant is used to replace, repair, or simulate a defective or damaged part of the human body and can be fully or partially implanted in the human body. For example, fibrous tissue wounds such as muscle, ligament and meniscus tears can be repaired with sutures. To simplify wound closure and improve immobilization, a doctor can insert the implant into the human body with a suturing device to help the wound heal.

It should be noted that the “front end” and “rear end” involved in some embodiments of present disclosure refer to an end point, an end surface, an end portion, a portion close to the end portion and having a certain length of the implant or the suture device. The “upper end” and “lower end” refer to a direction of the implant or the suturing device during placement or operation. The axial direction refers to an axis along a length direction of the implant or the suturing device. The circumferential direction refers to a direction around a central region (e.g. around the axis). For example, a direction of an end of the puncture needle of the suturing device that penetrates into the human body is the “front end”; correspondingly, the “rear end” refers to a direction of an end of the suturing device away from the human body along the axial direction of the puncture needle. As another example, after the puncture needle penetrates into the human body, a direction of an end that adheres to a human cell tissue is the lower end; correspondingly, the “upper end” refers to a direction away from the human body. As yet another example, the axial direction of the puncture needle refers to an axis of the puncture needle along a length direction of the puncture needle; the circumferential direction of the implant refers to a direction perpendicular to the length direction of the implant; the axial direction of the suturing device refers to an axis along the length direction of the suturing device.

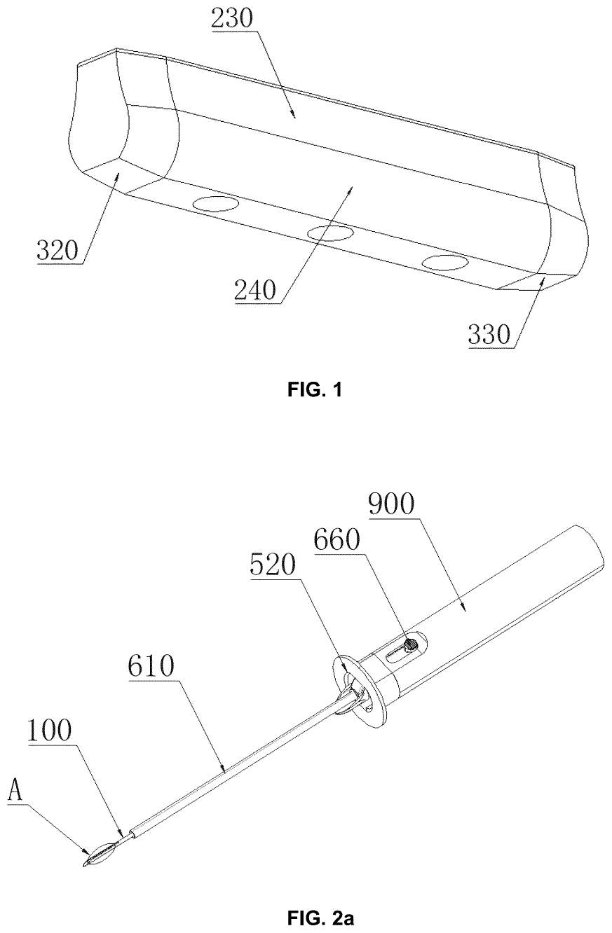

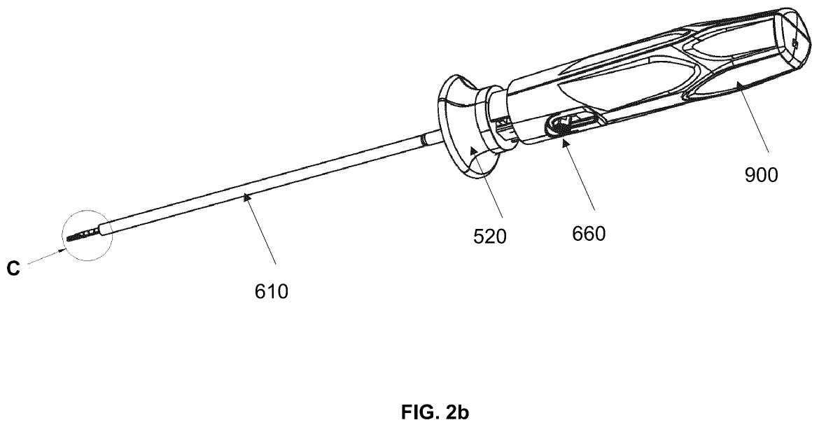

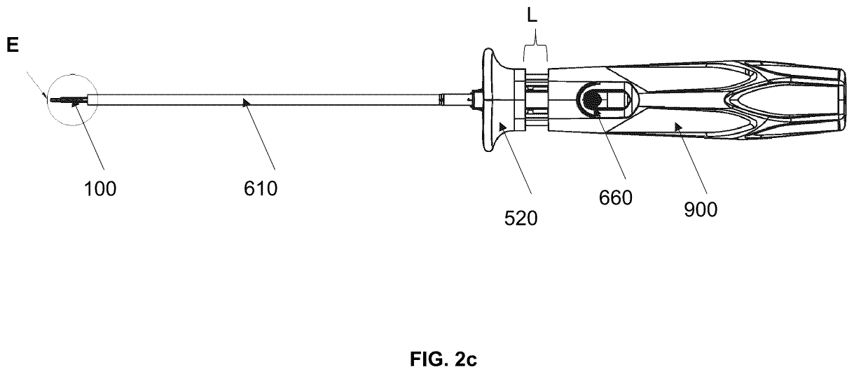

FIG. 1 is a schematic diagram illustrating an implant according to some embodiments of the present disclosure. FIG. 2a is a schematic diagram illustrating a suturing device according to some embodiments of the present disclosure; FIG. 2b is a schematic diagram illustrating a suturing device according to some embodiments of the present disclosure; FIG. 2c is a schematic diagram illustrating a top view of a suturing device according to some embodiments of the present disclosure. FIG. 3a is a schematic diagram illustrating a partial enlargement of a position A in FIG. 2a; FIG. 3b is a schematic diagram illustrating a partial enlargement of a position C in FIG. 2b; FIG. 3c is a schematic diagram illustrating a partial enlargement of a position E in FIG. 2c.

In some embodiments, as shown in FIG. 1, a first chamfer 320 is provided between a front end surface of the implant and a circumferential sidewall of the implant; and a second chamfer 330 is provided between a rear end surface of the implant and the circumferential sidewall of the implant. In some embodiments, a plurality of circumferential sidewalls of the implant are provided with the first chamfer 320, respectively. In some embodiments, the first chamfer 320 is provided in one or more circumferential sidewalls of the implant except an upper end sidewall. An upper end of the implant refers to a side of the implant away from a human body after the implant is placed in a suturing device when the suturing device is used.

In some embodiments of the present disclosure, when a puncture needle 100 penetrates into the human body, the puncture needle 100 may bend slightly. The first chamfer 320 and the second chamfer 330 can help avoid an inner wall causing the puncture needle to bend, thereby preventing the puncture needle 100 from immediately contacting an end portion of the implant after the puncture needle bends slightly to cause the implant to break.

Some embodiments of the present disclosure further provide a suturing device. The suturing device may include the puncture needle 100 and one or more implants described in some embodiments. In some embodiments, as shown in FIGS. 1-3c, the one or more implants are disposed on the puncture needle 100; the puncture needle 100 is provided with a channel 110 extending along an axial direction, and the one or more implants are triggered from the suturing device through the channel 110.

The suturing device refers to a tool or device used by an operator (e.g., a doctor) to perform a suturing operation. The operator places the implant into the puncture needle and trigger the implant through the suturing device (e.g., trigger the implant through a triggering mechanism described below).

In some embodiments, the puncture needle 100 may include a hollow hole. A notch communicated with the hollow hole may be disposed on a circumferential sidewall of the puncture needle 100. The notch and the hollow hole may enclose to form a slot structure. The slot structure may form the channel 110.

In some embodiments, the implant may include a first portion 230 and a second portion 240. When the implant is placed in the puncture needle, the first portion 230 may be located at an upper end of the puncture needle, and the second portion 240 may be located at a lower end of the puncture needle. In some embodiments, the implant may include the first portion 230 and the second portion 240 which are connected in sequence from top to bottom along a depth direction of the slot structure. A suspension structure may be provided on a sidewall of the first portion 230, and the first portion 230 may be suspended on the notch through the suspension structure, such that a clearance is formed between a bottom of the second portion 240 and an inner sidewall of the hollow hole. The second portion 240 may be located in the hollow hole, and a width at a widest position of the second portion 240 may be greater than a width of the notch.

The suspension structure may be in various forms. For example, the implant may be a structure with a narrow upper portion and a wide lower portion, and a shape of a cross-section of the first portion 230 of the implant may be approximately rectangular; and the suspension structure may be provided at a position close to a top surface of the implant, the suspension structure may be a convex edge protruding to two sides, and the convex edge may be suspended outside the notch. As another example, the cross-section of the first portion may be an inverted trapezoid with a wide upper portion and a narrow lower portion, and a width at a widest position of the first portion may be greater than the width of the notch, such that the implant is suspended without falling and the bottom of the implant is prevented from contacting the bottom of the hollow hole. In some embodiments, the first portion 230 may be slidably connected to the notch of the puncture needle to prevent the implant from rotating and dislocating during a sliding process. In some embodiments, a shape of a cross-section of the second portion 240 may be an approximately incomplete spherical structure.

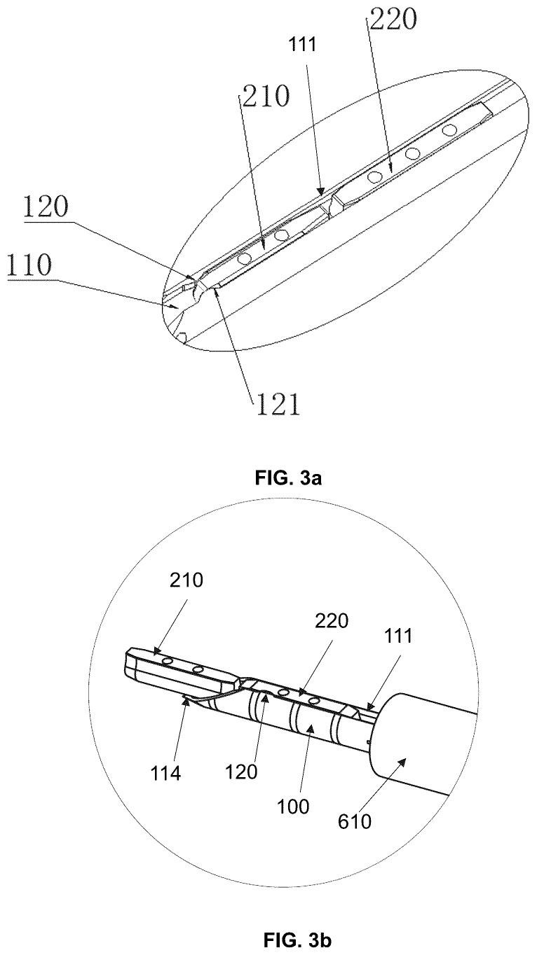

In some embodiments, a bottom surface of the implant may be a plane. For example, the implant may be molded, and the bottom surface of the implant may be a plane during molding. In some embodiments, a bottom surface of the second portion 240 may be processed into a plane to increase a distance from a bottom surface of the circular hollow hole of the puncture needle, so as to prevent the puncture needle 100 from immediately contacting the bottom surface of the implant after the puncture needle 100 bends slightly to cause breakage of the implant. In some embodiments, the bottom surface of the second portion 240 may be in a shape that can form a deformation clearance (e.g., a deformation clearance 310 in FIG. 4) with the bottom surface of the puncture needle 100. The shape may include a regular shape or an irregular shape. For example, the bottom surface of the second portion 240 may be a concave curved surface, a serrated surface, an irregular shape matching the bone or tissue at the wound of the human body, etc., and the deformation clearance may be formed between the bottom surface of the second portion 240 and the bottom surface of the puncture needle. After the puncture needle 100 bends slightly, the deformation clearance can prevent the puncture needle 100 from immediately contacting the bottom surface of the implant, thereby preventing the implant from being squeezed by the puncture needle 100 and thus broken.

In some embodiments, the first chamfer 320 and the second chamfer 330 may be within a range of 8°-10°, respectively. For example, the first chamfer 320 and the second chamfer 330 may be 8°, 9°, or 10°, etc. An angle of the first chamfer 320 and the second chamfer 330 refers to an angle between a chamfered surface and an original circumferential sidewall or a surface of the implant. The larger the angle of the first chamfer and the second chamfer, the better the avoidance effect. If the angle is too large, the strength of the implant may decrease. When the first chamfer 320 and the second chamfer 330 are within the range of 8°-10°, the avoidance effect and the strength of the implant can both be considered. In some embodiments, the implant may be provided with a hole. The hole may be used as a suturing path for the suturing device; and a suture may pass through the hole.

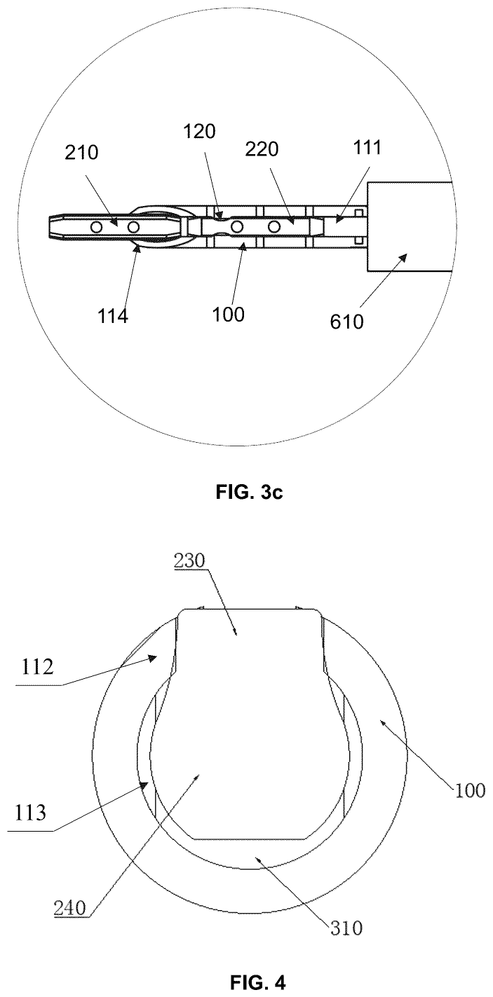

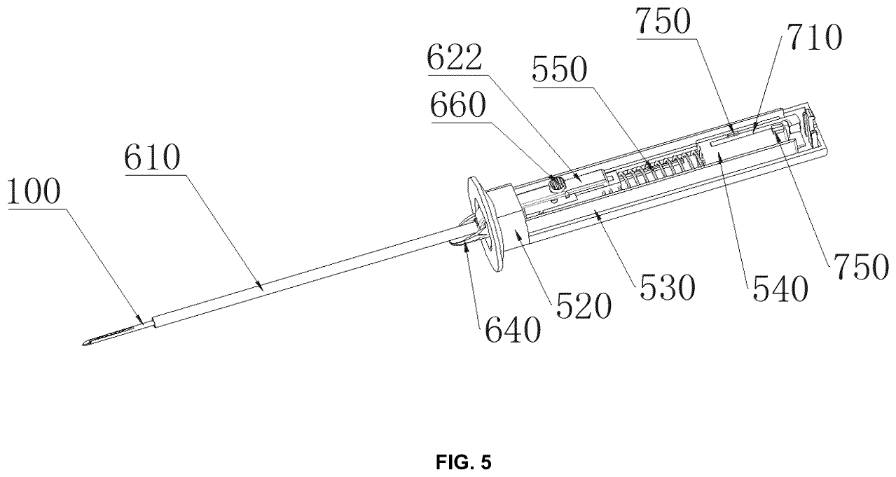

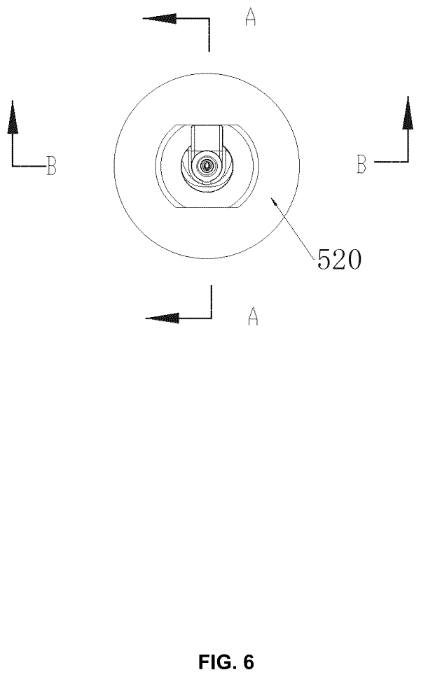

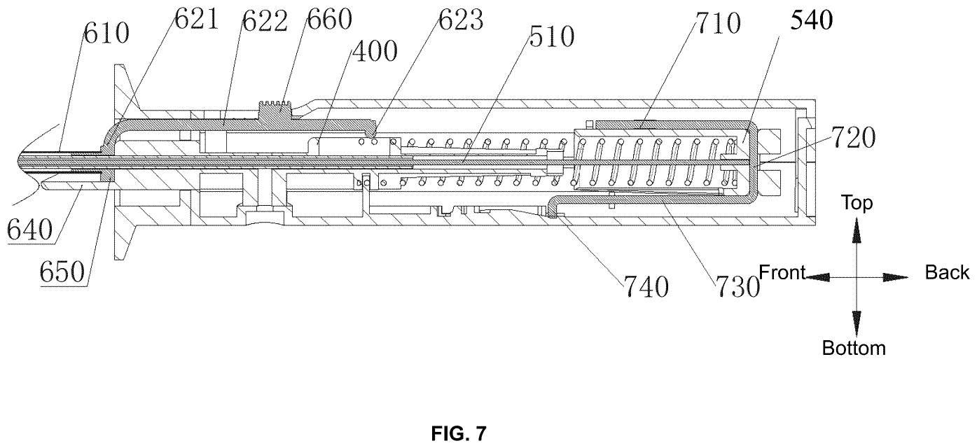

FIG. 4 is a schematic diagram illustrating a cross-section of a puncture needle of a suturing device according to some embodiments of the present disclosure. FIG. 5 is a schematic diagram illustrating a suturing device excluding an upper half housing according to some embodiments of the present disclosure. FIG. 6 is a schematic diagram illustrating a front view of a suturing device according to some embodiments of the present disclosure. FIG. 7 is a schematic diagram illustrating a cross-sectional view in an A-A direction in FIG. 6. FIG. 8 is a schematic diagram illustrating a cross-sectional view in a B-B direction in FIG. 6.

In some embodiments, the suturing device may include a puncture needle and one or more implants. A first chamfer may be provided between a front end surface of each of the one or more implants and a circumferential sidewall of the implant, and a second chamfer may be provided between a rear end surface of each of the one or more implants and the circumferential sidewall of the implant.

In some embodiments, as shown in FIG. 2, the suturing device may include the puncture needle 100 and a plurality of implants. A count of implants may be two, three, or more. In some embodiments of the present disclosure, two implants may be taken as an example. In some embodiments, as shown in FIG. 2a, the plurality of implants are disposed on the puncture needle 100; the puncture needle 100 is provided with the channel 110 extending along an axial direction, the plurality of implants are arranged in the channel 110, and the plurality of implants are triggered from the suturing device after passing through the channel 110 in sequence.

In some embodiments, at least two limiting structures 120 are provided on an inner wall of the channel, and the at least two limiting structures 120 are arranged along a circumferential direction. The at least two limiting structures 120 may at least block a second implant to be triggered, such that an applied force may be increased when the second implant to be triggered is triggered from the suturing device. The at least two limiting structures 120 may include protrusions, bosses, retaining rings, etc. For example, the at least two limiting structures 120 may be retaining rings provided on the inner wall of the channel 110. In some embodiments, the at least two limiting structures 120 may include inward protrusions provided on the inner wall of the channel 110. When the implant is triggered to move to the position of the limiting structures, the protrusions may contact a surface of the implant to block the implant.

In some embodiments, the at least two limiting structures 120 may block the one or more implants. For example, the at least two limiting structures 120 may block all the implants. As another example, when a plurality of implants are provided, the at least two limiting structures 120 may block some of the implants. In some embodiments, the at least two limiting structures 120 are located on a triggering path of the implant. In some embodiments, the at least two limiting structures 120 are disposed on a triggering path from the second implant to be triggered to a front end of the puncture needle 100. For example, when two implants are arranged in the puncture needle, one of the two implants close to the front end of the puncture needle 100 is the first implant to be triggered, and the other of the two implants away from the front end of the puncture needle 100 is the second implant to be triggered. The at least two limiting structures 120 may be disposed at a position between the second implant to be triggered (hereinafter referred to as a second implant) and the first implant to be triggered (hereinafter referred to as a first implant). The at least two limiting structures 120 can at least block a second implant 220. Under the blocking effect of the at least two limiting structures 120, a friction force on the second implant 220 is increased, and it is more difficult for the second implant 220 to pass through the position where the at least two limiting structures 120 are located; the operator or the suturing device needs to apply a greater external force to the second implant 220 to make the second implant 220 pass through the at least two limiting structures 120, thereby reducing the risk of the first implant 210 bringing the second implant 220 out.

It should be noted that the at least two limiting structures 120 play a role in blocking rather than stopping. The second implant 220 can still pass through the at least two limiting structures 120, but it is more difficult to pass through the position in the channel 110 where the at least two limiting structures 120 are provided than the position in the channel 110 where no two limiting structure 120 is provided.

In some embodiments, a plurality of limiting structures 120 are provided in a circumferential direction of the puncture needle 100. In the circumferential direction, two or more limiting structures 120 are provided. When the implant passes through the position where the limiting structures 120 are located, the plurality of limiting structures are in sliding contact with the circumferential sidewall of the implant simultaneously to increase a friction force on the implant, thereby achieving a better blocking effect. In some embodiments, in the circumferential direction, an even count of limiting structures 120 may be provided and arranged opposite to each other in pairs. For example, two, four, or six limiting structures 120 may be provided, and half of the limiting structures 120 may be arranged on each of the two sides of the notch of the puncture needle 100. That is, one, two, or three limiting structures may be arranged on each of the two sides of the notch of the puncture needle 100.

In some embodiments, as shown in FIG. 3a, each of the limiting structures 120 may include a guide slope 121 obliquely arranged relative to the inner wall of the channel 110. The implant may be in sliding contact with the guide slope 121. The guide slope 121 may be configured to guide the implant to cross over the limiting structures 120.

Merely by way of example, the limiting structures 120 may be wedge-shaped or hemispherical, and a smoothly transition may be formed between a rear edge of the guide slope 121 and the inner wall of the channel 110; from back to front, the guide slope 121 may gradually tilt toward a centerline of the channel 110, and the guide slope 121 may be a plane or a curved surface. The implant may move forward from a rear side of the limiting structures 120 and first contact the guide slope 121. Under the action of the guide slope 121, a pressure of the limiting structures 120 on the implant gradually increases, and the friction force on the implant gradually increases, such that a greater external force needs to be applied to the second implant 220 to make the second implant 220 pass through the limiting structures 120.

In some embodiments, the limiting structure 120 may block the one or more implants. For example, the limiting structures 12 may block all the implants. That is, all the implants, especially the first implant 210, may be blocked by the limiting structures 120, thereby preventing the first implant 210 from slipping out of the puncture needle 100. All the implants may have exactly the same shape, which makes the preparation of the implants more convenient and more versatile.

In some embodiments, the first implant may be provided with an avoidance structure for avoiding the limiting structures 120, such that the friction force on the first implant when passing through the limiting structures may be less than the friction force on the second implant when passing through the limiting structures, and the first implant may pass through the limiting structures 120 more easily than the second implant. In some embodiments, the friction force on the first implant 210 caused by the limiting structures 120 is relatively small, such that the blocking effect on the first implant is relatively small. In some embodiments, the avoidance structure may include an avoidance slot. The avoidance slot may be disposed at a position of the first implant 210 opposite to the limiting structures 120. In some embodiments, the avoidance slot may connect a front end and a rear end of the first implant 210 along the axial direction. When the first implant 210 passes through the position where the limiting structures 120 are located, the limiting structures 120 may slide through the avoidance slot. In some embodiments, the avoidance structure may include that an outer diameter of the first implant 210 is less than an outer diameter of the second implant 220, thereby reducing a contact area between the limiting structures 120 and the first implant 210, reducing the friction force, and facilitating the triggering of the first implant.

In some embodiments, a of the first implant 210 may be different from a size of the second implant 220. In some embodiments, a length of the first implant 210 may be within a range of 4.9 mm-5.5 mm, a width of the first implant 210 may be within a range of 0.9 mm-1.3 mm, and a height of the first implant 210 may be within a range of 1.1 mm-1.5 mm. In some embodiments, a length of the second implant 220 may be within a range of 5.2 mm-5.8 mm, a width of the second implant 220 may be within a range of 0.9 mm-1.3 mm, and a height of the second implant 220 may be within a range of 1.1 mm-1.5 mm. In some embodiments, the length of the first implant 210 may be within a range of 4.8 mm-5.4 mm, the width of the first implant 210 may be within a range of 0.8 mm-1.5 mm, and the height of the first implant 210 may be within a range of 1.0 mm-1.6 mm. In some embodiments, the length of the second implant 220 may be within a range of 5.1 mm-5.9 mm, the width of the second implant 220 may be within a range of 0.8 mm-1.5 mm, and the height of the second implant 220 may be within a range of 1.0 mm-1.6 mm. The length refers to a size in a front-back direction in FIG. 7; the height refers to a size in a vertical direction in FIG. 7. By limiting the size of the first implant 210 and the size of the second implant 220, the first implant 210 and the second implant 220 can be triggered smoothly, and the limiting structures 12 have a greater blocking effect on the second implant 220.

In some embodiments, as shown in FIGS. 3a-FIG. 4, the puncture needle 100 may include a hollow hole, and a notch 111 communicated with the hollow hole may be provided on the circumferential sidewall of the puncture needle 100. In the axial direction, a front end of the notch 111 may be communicated with an end surface of a tip 114 of the puncture needle 100. The notch 111 and the hollow hole may enclose to form a slot structure 112. The slot structure 112 may form the channel 110. Each of the limiting structures 120 may include a protrusion located on an inner surface of the slot structure.

The implant may be located in the slot structure. When the implant slides along the slot structure and contacts the protrusions of the limiting structures 120, slot walls may drive the protrusions to press against the sidewall of the implant, thereby increasing the friction force and achieving the blocking effect on the second implant. Under the expansion of the implant, the two slot walls of the slot structure may slightly deform to increase a width of the notch of the slot structure, such that the second implant may pass through the limiting structures 120 without being blocked. The slot structure can more conveniently achieve the blocking effect on the second implant using the deformation of the slot structure, which makes the structure simpler.

In some embodiments, as shown in FIG. 4, protrusions 113 are disposed on two opposite sidewalls of the slot structure 112. The two protrusions 113 may be disposed opposite to each other, and the two protrusions 113 may be a hemispherical structure, respectively.

In some embodiments, as shown in FIG. 3a and FIG. 4, at least a portion of the one or more implants may be located in the hollow hole. When the portion of the one or more implants located in the hollow hole abuts against a notch of the slot structure, a deformation clearance 310 may be formed between a bottom surface of the one or more implants and a bottom surface of the slot structure. In some embodiments, when a plurality of implants are located in the hollow hole, the deformation clearance 310 may be formed between a bottom surface of each of the implants and the bottom surface of the slot structure.

In general, the operator makes a slope of the tip 114 of the puncture needle 100 point upward and then inserts the puncture needle 100 downward for puncturing. During the puncturing, when the puncture needle 100 is blocked, the puncture needle 100 may bend slightly, and the bent channel 110 may gradually approaches the bottom surface of the implant. Since the clearance is reserved between the bottom surface of the implant and the bottom surface of the slot structure, the inner wall of the bent puncture needle 100 can be prevented from immediately contacting the implant to cause the implant to break.

In some embodiments, the deformation clearance 310 may be within a range of 0.05 mm-0.4 mm. In some embodiments, the deformation clearance 310 may be within a range of 0.1 mm-0.2 mm. In some embodiments, the deformation clearance 310 may be within a range of 0.05 mm-0.3 mm. In some embodiments, the deformation clearance 310 may be within a range of 0.2 mm-0.4 mm. In some embodiments, the deformation clearance 310 may be within a range of 0.15 mm-0.5 mm. In some embodiments, the range of the deformation clearance may be determined based on a structure, a size, and a material of the puncture needle. For example, when a diameter of the puncture needle is large, the deformation clearance may be 0.4 mm. As another example, when the diameter of the puncture needle is small, the deformation clearance may be 0.15 mm.

In some embodiments, as shown in FIG. 5, the suturing device may include a fixing portion 400 and a triggering mechanism. The puncture needle 100 may be connected with the fixing portion 400. The triggering mechanism may move along an axial direction relative to the fixing portion 400. The triggering mechanism may be configured to trigger one or more implants toward a front end. In some embodiments, when the one or more implants are triggered, the puncture needle 100 and the fixing portion 400 may not move, and the triggering mechanism may push the one or more implants to move along the axial direction of the puncture needle 100, such that the one or more implants may move to the front end of the puncture needle 100.

In some embodiments, the suturing device may further include a housing 900. A portion of the fixing portion 400 may be located inside the housing 900 and fixedly connected to the housing 900. The puncture needle 100, the fixing portion 400, and the housing 900 may be relatively fixed.

In some embodiments, the triggering mechanism may push the one or more implants located on the puncture needle 100 forward through a push pin 510. In some embodiments, as shown in FIG. 5, FIG. 7 and FIG. 8, the triggering mechanism may include the push pin 510, a push button 520, a connection arm 530, and a linkage portion 540. The push button 520, the connection arm 530, and the linkage portion 540 may be arranged in sequence along a direction from a front end of the suturing device to a rear end of the suturing device. A front end of the push pin 510 may be located in the hollow hole of the puncture needle 100. The push button 520 may be slidably connected with the fixing portion 400. The push button 520 may move in a front-back direction. A front end of the connection arm 530 may be connected with the push button 520, and a rear end of the connection arm 530 may be connected with the linkage portion 540. The linkage portion 540 may be connected with a rear end of the push pin 510. In some embodiments, the push button 520, the connection arm 530, the linkage portion 540, and the push pin 510 may be fixedly connected and have the same movement state.

In some embodiments, the connection arm 530 may connect the push button 520 and the linkage portion 540 as a whole, and the push pin 510 may be located inside the whole formed by connection of the connection arm 530, the push button 520, and the linkage portion 540. When any one of the push button 520, the connection arm 530, or the linkage portion 540 is pushed forward relative to the housing 900, a forward thrust may be transmitted to the linkage portion 540 and the push pin 510 to make the push pin 510 move forward. After the push button 520 is pushed forward relative to the housing 900, the linkage portion 540 may drive the push pin 510 to move forward, thereby pushing the implant in the channel 110 forward until pushing the implant out of the puncture needle 100. In some embodiments of the present disclosure, by driving the push pin to move through the connection arm 530 and the linkage portion 540, the push button can be disposed at a position convenient for the operator to control rather than necessarily being disposed at the rear end of the push pin, such that the structure of the suturing device is more in line with human mechanics. For example, when a person holds the suturing device with a hand, the push button is located at the position of the thumb, which is convenient for human operation.

In some embodiments, a spring 550 is provided between the fixing portion 400 and the linkage portion 540. When the push button 520 moves forward, the linkage portion 540 may squeeze the spring 550. When an external force on the push button 520 is released, the spring 550 may be configured to drive the linkage portion 540 to move in a direction away from the fixing portion 400, thereby resetting the push button 520. In some embodiments, two ends of the spring 550 may be fixed to the fixing portion 400 and the linkage portion 540, respectively.

In some embodiments, the suturing device may further include a depth limiting tube 610 and a locking mechanism. The depth limiting tube 610 may sleeve an outer side of the puncture needle 100, and a front end of the depth limiting tube 610 may be a depth limiting end. A position of the depth limiting tube 610 in the axial direction is adjustable. The locking mechanism may be configured to lock the depth limiting tube 610 such that the position of the depth limiting tube 610 may be fixed relative to the puncture needle 100. In some embodiments, the position of the depth limiting tube 610 in the axial direction being adjustable means that a distance between the depth limiting tube 610 and the front end of the puncture needle 100 can be adjusted.

In some embodiments, the depth limiting tube 610 sleeves the outer side of the puncture needle 100, and the front end of the depth limiting tube 610 may be a limiting end. By sliding the depth limiting tube 610, the distance between the depth limiting tube 610 and the front end of the puncture needle 100 can be changed, thereby changing a length of the puncture needle 100 exposed to the outside. Before suturing, a thickness of a tissue to be sutured may be measured first, and then the position of the depth limiting tube 610 may be adjusted and locked such that a length of a portion of the puncture needle 100 exposed at the front end of the depth limiting tube 610 matches the thickness of the tissue to be sutured. During the suturing process, the limiting end may abut against a surface of the tissue.

In some embodiments, as shown in FIG. 7 and FIG. 8, the locking mechanism may include a control arm. The control arm may include a first connection arm 621, a pressing arm 622, and a second connection arm 623 which are connected in sequence along the axial direction. In some embodiments, the first connection arm 621 and the second connection arm 623 may be arranged at an angle with the pressing arm 622, respectively, and the control arm may be approximately in an “inverted U” shape. One end (e.g., a lower end) of the first connection arm 621 may be fixedly connected with the depth limiting tube 610, and the other end (e.g., an upper end) of the first connection arm 621 may be connected with one end (e.g., a front end) of the pressing arm 622. One end (e.g., an upper end) of the second connection arm 623 may be connected with the other end (e.g., a rear end) of the pressing arm 622, and the other end (e.g., a lower end) of the second connection arm 623 may be slidably connected with the fixing portion 400. In some embodiments, the other end of the second connection arm 623 may abut against the fixing portion 400 and may slide along the axial direction relative to the fixing portion 400. In some embodiments, since the distance between the fixing portion 400 and an axis of the puncture needle is greater than a radius of the depth limiting tube 610, a distance between the first connection arm 621, the second connection arm 623 and the pressing arm 622 and the axis of the puncture needle 100 satisfies: a distance between the pressing arm 622 and the axis of the puncture needle 100>a distance between the second connection arm 623 and the pressing arm 622 and the axis of the puncture needle>a distance between the first connection arm 621 and the axis of the puncture needle.

In some embodiments, a clearance is provided between the pressing arm 622 and the fixing portion 400 such that the pressing arm 622 is capable of moving relative to the fixing portion 400. In some embodiments, a gear pin 6221 is provided on the pressing arm 622. The locking mechanism may further include a plurality of limiting slots 630 located on the housing 900. The plurality of limiting slots 630 are arranged in sequence along the axial direction. The pressing arm 622 has a locked state and an unlocked state. In the locked state, the gear pin 6221 is located in one of the limiting slots 630, and the limiting slot 630 is configured to prevent the pressing arm 622 from moving along the axial direction. In the unlocked state, the gear pin 6221 is pressed out of the limiting slot 630, and the pressing arm 622 is capable of moving along the axial direction. In some embodiments, the operator may the pressing arm 622 toward the fixing portion 400 to cause the pressing arm 622 deform, the gear pin 6221 may be pressed out of the limiting slot 630, the pressing arm 622 may be switched to the unlocked state, and the pressing arm 622 is capable of moving along the axial direction. After the external force on the pressing arm 622 is released, the pressing arm 622 may return to an original state, and the gear pin 6221 may enter the limiting slot 630 corresponding to the position of the gear pin 6221 again. Through the cooperation between the gear pin 6221 and the limiting slots 630, a relative position of the depth limiting tube 610 and the puncture needle 100 can be conveniently adjusted.

In some embodiments, as shown in FIG. 7, a slide slot corresponding to the position of the pressing arm 622 may be provided on the housing 900. The slide slot connects the inside and outside of the housing 900. A button 660 may be connected to the pressing arm 622, and the button 660 may be configured to slide along the slide slot. By pressing the button 660 and pushing and pulling the button 660, the position of the depth limiting end can be changed and locked.

In some embodiments, a first guide slot 640 extending along the axial direction is provided on the fixing portion 400. A sliding abutting portion 650 is provided on the first connection arm 621. The sliding abutting portion 650 may abut against the first guide slot 640. The first guide slot 640 may be configured to guide the first connection arm 621 to slide along the axial direction of the puncture needle 100.

In some embodiments, after the pressing arm 622 is subjected to a pressure forcing the pressing arm 622 to deform toward the fixing portion 400, a bottom end of the second connection arm 623 may abut against the fixing portion 400. In some embodiments, the first guide slot 640 may be an outer edge of the fixing portion 400 extending toward the front end. The sliding abutting portion 650 may be a support foot provided at a bottom end of the first connection arm 621 extending downward. The support foot may abut against the outer edge. By providing the first guide slot 640, the first connection arm 621 can always move along the axial direction during a forward and backward sliding process without rotating; and the first connection arm 621 can be supported by the first guide slot 640.

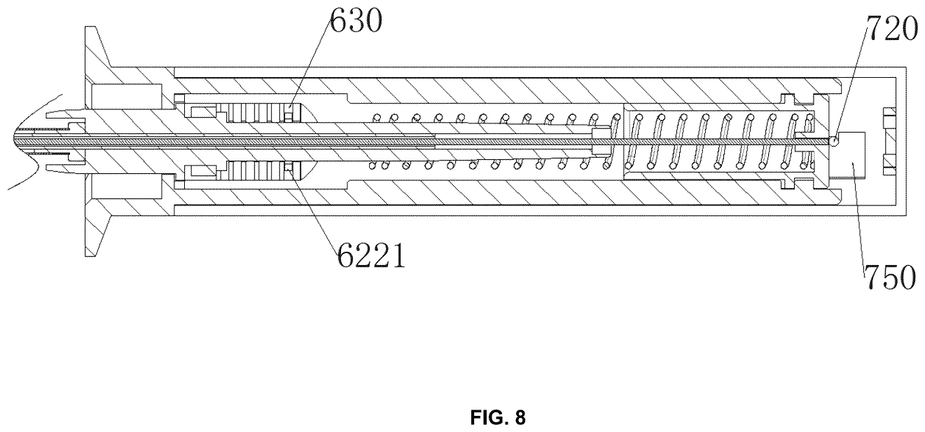

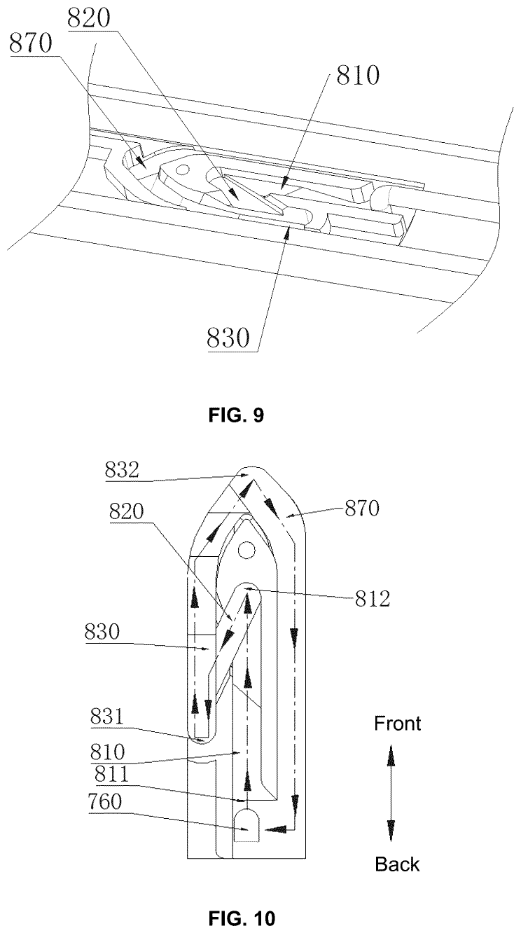

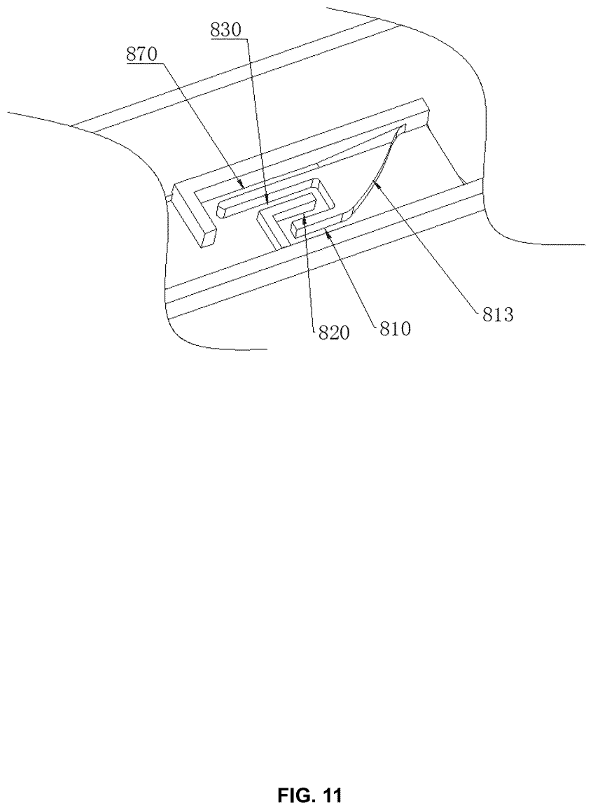

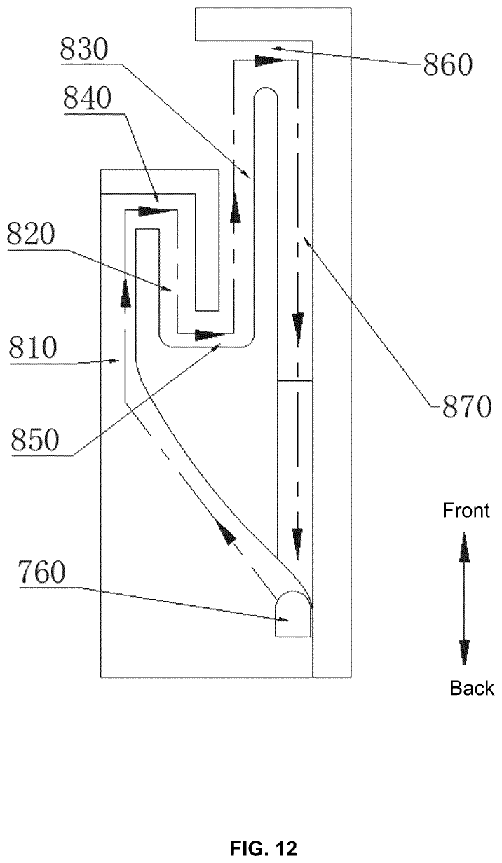

FIG. 9 is a schematic diagram illustrating a gear slot of a suturing device according to some embodiments of the present disclosure. FIG. 10 is a schematic diagram illustrating a movement path of a free end in the gear slot in FIG. 9. FIG. 11 is a schematic diagram illustrating another gear slot of a suturing device according to some embodiments of the present disclosure. FIG. 12 is a schematic diagram illustrating a movement path of a free end in the gear slot in FIG. 11.

In some embodiments, as shown in FIGS. 9-12, the suturing device may include the housing 900 and a triggering mechanism. The triggering mechanism may be configured to trigger an implant toward a front end (i.e., in a direction close to the tip 114 of the puncture needle). A gear slot is provided on an inner wall of the housing 900. An elastic swing rod is provided on the triggering mechanism. A fixed end of the elastic swing rod may be fixedly connected with the triggering mechanism, and a free end 760 (see FIG. 10) of the elastic swing rod may extend from a notch of the gear slot into the gear slot. Merely by way of example, the gear slot may include a plurality of slots. When the free end 760 of the elastic swing rod extends into one of the slots, the triggering mechanism may be limited to a gear corresponding to the slot to control a position of the triggering mechanism in an axial direction.

In some embodiments, the triggering mechanism may be located on a side of the notch of the gear slot. The triggering mechanism may move in a front-back direction (e.g., a front-back direction shown in FIG. 7) along the axial direction. The elastic swing rod may move with the triggering mechanism. The elastic swing rod may include an elastic metal rod. One end of the elastic swing rod may be fixed to the triggering mechanism, and the other end of the elastic swing rod may be the free end 760. The free end 760 may extend into the gear slot. In some embodiments, during the movement of the triggering mechanism, the elastic swing rod may swing in a plane perpendicular to an opening of the gear slot, and may move toward or away from a bottom surface of the gear slot, such that the free end 760 may move along an extension path of the gear slot, thereby helping the operator to control a gear of the triggering mechanism. In some embodiments, a plane where the opening of the gear slot is located may be a plane parallel to B-B in FIG. 6; a plane perpendicular to the plane where the opening of the gear slot is located may be the plane shown in FIG. 7. That is, the elastic swing rod may swing up and down, and a swing direction of the elastic swing rod be forward approaching or backward away from the bottom surface of the gear slot.

In some embodiments, as shown in FIG. 7 and FIG. 8, the elastic swing rod is connected to the linkage portion 540. The elastic swing rod may include a first longitudinal rod 710, a first vertical rod 720, a second longitudinal rod 730, and a second vertical rod 740 which are connected end to end in sequence. The first longitudinal rod 710 and the second longitudinal rod 730 may be parallel to the axial direction. A rear end of the first longitudinal rod 710 may be connected with an upper end of the first vertical rod 720. A lower end of the first vertical rod 720 may be connected with a rear end of the second longitudinal rod 730. An upper end of the second vertical rod 740 may be connected with a front end of the second longitudinal rod 730. A lower end of the second vertical rod 740 may be the free end 760. The first vertical rod 720 may be located on a surface of the linkage portion 540 away from the gear slot. The first vertical rod 720 may be located on a rear end surface side of the linkage portion 540. The second vertical rod 740 may extend into the gear slot.

In some embodiments, the connection between each rod and the linkage portion 540 may be achieved in various ways, such as snap connection, sleeve connection, etc. For example, the first longitudinal rod 710 and the first vertical rod 720 may be fixed to the linkage portion 540 through a limiting buckle 750. The limiting buckle 750 may be configured to prevent the first longitudinal rod 710 and the first vertical rod 720 from swinging relative to the linkage portion, respectively. In some embodiments, the limiting buckle 750 may also be provided on a surface of the linkage portion 540 facing the gear slot. The limiting buckle 750 may be close to the second longitudinal rod 730, and one end of the limiting buckle 750 away from the second vertical rod 740 may limit the second longitudinal rod 730. The second longitudinal rod 730 may swing with the limiting buckle 750 as a fulcrum. Merely by way of example, the limiting buckle 750 may be an arc slot arranged near an axial position of the puncture needle. The second longitudinal rod 730 may be a circular rod that is inserted into the arc slot. The circular rod may swing with the arc slot as a fulcrum, but the relative position of the second longitudinal rod 730 and the limiting buckle 750 in the axial direction remains constant.

In some embodiments, an avoidance slot may be provided on the surface of the linkage portion 540 facing the gear slot to form a sufficient space for swinging above the second longitudinal rod 730. The second longitudinal rod 730 may swing in the plane perpendicular to the opening of the gear slot, or may approach the gear slot or move away from the gear slot in the direction of the opening of the gear slot.

In some embodiments, as shown in FIG. 9 and FIG. 10, two implants may be provided in the suturing device. The gear slot may include a first slot unit 810 and a second slot unit 830. The gear slot may also include a first retreat slot 820. A bottom surface of the first retreat slot 820 may be lower than a bottom surface of the first slot unit 810. One end of the first retreat slot 820 may be located on a bottom surface of a first limiting end 812 of the first slot unit, and may extend from the first limiting end 812 toward the rear end and pass through a side wall of the first slot unit 810 to communicate with the second slot unit 830. An extension direction of the first retreat slot 820 may be arranged at an angle to an extension direction of the first slot unit 810. A bottom surface of the second slot unit 830 may be lower than the bottom surface of the first retreat slot 820. The extension direction of the first retreat slot 820 being arranged at an angle to the extension direction of the first slot unit 810 means that the first retreat slot 820 may not parallel to the first slot unit 810, such that the free end 760 may be restricted by an end surface during movement. The bottom surface of the second slot unit 830 may be lower than the bottom surface of the first retreat slot 820. When the free end 760 retreats to an end of the first retreat slot 820, the free end 760 may directly enter an initial position of the second slot unit 830.

In some embodiments, at the initial position, the free end 760 may be located at a first starting end 811, and under an elastic force of the elastic swing rod, a lower end surface of the free end 760 may abut against the bottom surface of the first slot unit 810. The push button 520 may be pushed forward to drive the linkage portion 540 to move forward. The free end 760 may move along the first slot unit 810 to the first limiting end 812, the free end 760 may be blocked, and the operator knows that the first implant 210 is triggered. An end portion of the first retreat slot 820 may be located at the first limiting end 812, and the bottom surface of the first retreat slot 820 may be lower than the bottom surface of the first slot unit 810. That is, when the first implant 210 is triggered and the free end 760 moves to the first limiting end 812, the lower end surface of the free end 760 may perform a step-down movement under the elastic force of the elastic swing rod, and the free end 760 may enter the first retreat slot 820 and abut against the bottom surface of the first retreat slot 820. When the push button 520 is pushed backward, the free end 760 may be limited by a slot wall of the first retreat slot 820, and thus may move backward until moving to a connection between the first retreat slot 820 and the second slot unit 830. The free end 760 may perform another step-down movement, the free end 760 may enter the second slot unit 830, and the lower end surface of the free end 760 may abut against the bottom surface of the second slot unit 830. The push button 520 may be pushed forward again to make the free end 760 move to the second limiting end 832, thereby completing the triggering of the second implant 220.

In some embodiments, a transition (e.g. a transition between the first recess slot 820 and the second slot unit 830) of each slot may be set in various ways to achieve the step-down action, such as a height difference between the bottom surfaces, a descending slope, an ascending slope, etc.

In some embodiments, the gear slot may further include a second retreat slot 870. A bottom surface of the second retreat slot 870 may be lower than the bottom surface of the second slot unit 830. One end of the second retreat slot 870 may be located on a bottom surface of a second limiting end 832 of the second slot unit 830. The second retreat slot 870 may extend toward the rear end along the second limiting end 832 and may be connected with the first starting end 811 of the first slot unit 810.

In some embodiments, when the free end 760 moves to the second limiting end 832, the free end 760 may transition to the second retreat slot 870. When the operator pushes the push button 520 backward, the free end 760 may return to the first starting end 811 under the guidance of a slot wall of the second retreat slot 870. In the first slot unit 810, a bottom surface of the first starting end 811 may be lower than a bottom surface at a position between the first starting end 811 and the first limiting end 812. The bottom surface of the first starting end 811 of the first slot unit 810 may be an ascending slope, and a middle position of the first slot unit 810 may be a plane, so as to facilitate the free end 760 pushed out of the second retreat slot 870 to enter the first starting end 811 again.

In some embodiments, the ascending slope may be provided on the bottom surface of the second slot unit 830. The ascending slope may be located behind the second limiting end 832. The free end 760 may first perform an ascending movement and then fall into the second retreat slot 870. The ascending slope may facilitate the setting of the step-down structure at the transition of each slot.

In some embodiments, the free end 760 may be guided by the slot walls of the first slot unit 810, the first retreat slot 820, the second slot unit 830, and the second retreat slot 870 guide, so as to guide the free end 760 to move in each slot.

In some embodiments, as shown in FIG. 11 and FIG. 12, two implants may be provided in the suturing device. The gear slot may include the first slot unit 810 and the second slot unit 830.

In some embodiments, the first slot unit 810 may include a first slot wall and a second slot wall 813. In some embodiments, as shown in FIG. 11 and FIG. 12, an extension direction of the first slot wall may be parallel to the axial direction, and a width between the second slot wall 813 and the first slot wall may gradually decrease in a direction from the rear end to the front end.

In some embodiments, the second slot unit 830 may be located on a side of the second slot wall 813 of the first slot unit 810. The first retreat slot 820 may be provided between the first slot unit 810 and the second slot unit 830. A front end of the first retreat slot 820 may be connected with the first limiting end 812 of the first slot unit 810 through a first transverse slot 840, and a rear end of the first retreat slot 820 may be connected with the second starting end 831 of the second slot unit 830 through a second transverse slot 850. The second retreat slot 870 may be provided on the side of the second slot unit 830. The second slot unit 830 may be located between the first retreat slot 820 and the second retreat slot 870. A front end of the second retreat slot 870 may be connected with the second limiting end 832 of the second slot unit through a third transverse slot 860, and a rear end of the second retreat slot 870 may be connected with the second slot wall 813. A rear end bottom surface of the second retreat slot 870 may be higher than the bottom surface of the first starting end 811 of the first slot unit 810, so as to form a down-step structure at a connection point between the second retreat slot 870 and the first slot unit 810. In the initial state, the free end 760 may be located in the first slot unit 810 and located behind the opening of the rear end of the second retreat slot 870 and may abut against the second slot wall 813.

In some embodiments, in the initial state, the free end 760 may abut against the second slot wall 813 of the first slot unit 810. When the operator pushes the push button 520 forward, the free end 760 may move forward and always abut against the second slot wall 813, and the free end 760 may swing to store a part of elastic potential energy. When the free end 760 moves to the first limiting end 812, the free end 760 may swing into the first retreat slot 820 through the first transverse slot 840 under the driving of the elastic potential energy, and the free end 760 may abut against a slot wall in the first retreat slot 820 away from the first slot unit 810. The operator may push the push button 520 backward, and the free end 760 may move backward in the first retreat slot 820. When the free end 760 moves to the second transverse slot 850, the free end 760 may swing into the second slot unit 830 and abut against a sidewall of the second slot unit 830 away from the first slot unit 810 under the elastic action of the elastic swing rod. The push button 520 may be pushed forward, and the free end 760 may move to the second limiting end 832. The second implant 220 may be triggered. Meanwhile, the free end 760 may swing into the second retreat slot 870 through the third transverse slot 860. The push button 520 may be pushed backward, and the free end 760 may slide backward in the second retreat slot 870 and return to the first starting end 811.

In some embodiments, a first pin push distance may be greater than or equal to a sum of the length and a push clearance of the first implant 210. The first pin push distance refers to a distance required to trigger the first implant 210. The push clearance refers to a clearance between the push pin 510 and the second implant 220. A second pin push distance may be greater than or equal to the length of the second implant 220. The second pin push distance refers to a distance required to trigger the second implant 220. In some embodiments, the first pin push distance may be greater than the second pin push distance. In some embodiments, as shown in FIG. 10, the first pin push distance refers to an axial distance of the free end 760 moving from the first starting end 811 to the first limiting end 812; the second pin push distance refers to an axial distance of the free end 760 moving from the second starting end 831 to the second limiting end 832. In some embodiments, when the first implant 210 completes triggering, the push button 520 may not return to the initial position, but the push button 520 may protrude relative to the housing 900 (i.e., there is a clearance L between the push button 520 and the housing 900), which informs the operator that the first implant 210 completes triggering. Merely by way of example, FIG. 2c shows the position of push button 520 when the first implant 210 completes triggering. In this case, there is the clearance L between the push button 520 and the housing 900, and the clearance L informs the operator that the first implant 210 completes triggering. When the second implant 220 completes triggering, the gap L may continue to increase. When the free end 760 retreats to the first starting end 811 (i.e. the position of the initial state), the clearance L may be 0 or close to 0.

In some embodiments, a material of the housing 900 may include a transparent material. The position of the free end 760 may be clearly seen through the housing 900, which is convenient for the operator to control the suturing device and better observe the progress of puncturing. For example, the transparent material may include polycarbonate, polymethyl methacrylate, thermoplastic polyurethane, etc. In some embodiments, the shape of the housing 900 may be circular tube, a square tube, an irregular shape, etc. For example, as shown in FIG. 2a, the shape of the housing 900 is a circular tube. As another example, as shown in FIG. 2b and FIG. 2c, the shape of the housing 900 is an irregular shape easy to hold by the operator.

In some embodiments, the housing 900 may be formed by splicing an upper half housing and a lower half housing. The lower half housing and the fixing portion 400 may be fixedly connected by a threaded member. The threaded member may include a screw, a bolt, a nut, a stud, or the like.

In some embodiments, the suturing device may further include a suture (not shown in the figure). The suture may be configured to connect two implants. Merely by way of example, one end of the suture may be fixed to the first implant, and the other end of the suture may be a movable end and placed on the puncture needle 100.

In some embodiments, the puncture needle, the fixing portion, the triggering mechanism, the depth limiting tube, the locking mechanism, and the housing may be made of a metallic (e.g., stainless steel) and/or non-metallic material (e.g., a non-metallic biocompatible material). The implant may be made of a rigid, biocompatible material, such as polyethylene, acetal, or polypropylene. Alternatively, the implant may be made of a metallic, elastically deformable material, or a bioabsorbable material. The implant may be an integral injection molded part, and may also be manufactured by other processes. The connectors between the components may be formed by mechanical means, adhesive means (e.g., non-toxic, biocompatible adhesive glue) or other means known to those skilled in the art.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various parts described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. An implant, wherein a first chamfer is provided between a front end surface of the implant and a circumferential sidewall of the implant; and
a second chamfer is provided between a rear end surface of the implant and the circumferential sidewall of the implant.

2. The implant of claim 1, wherein a bottom surface of the implant is a plane.

3. The implant of claim 1, wherein the first chamfer and the second chamfer are within a range of 8°-10°, respectively.

4. A suturing device, comprising a puncture needle and one or more implants, wherein a first chamfer is provided between a front end surface of each of the one or more implants and a circumferential sidewall of each of the one or more implants, and a second chamfer is provided between a rear end surface of each of the one or more implants and the circumferential sidewall of each of the one or more implants; the one or more implants are disposed on the puncture needle; the puncture needle is provided with a channel extending in an axial direction, and the one or more implants are capable of being triggered from the suturing device through the channel; and
at least two limiting structures are disposed on an inner wall of the channel and arranged along a circumferential direction; the at least two limiting structures are capable of at least blocking a second implant to be triggered, such that an applied force is increased when the second implant to be triggered is triggered from the suturing device.

5. The suturing device of claim 4, wherein each of the at least two limiting structures includes a guide slope obliquely arranged relative to the inner wall of the channel, the one or more implants are in sliding contact with the guide slope, and the guide slope is configured to guide the one or more implants to cross over the at least two limiting structures.

6. The suturing device of claim 4, wherein the at least two limiting structures are capable of blocking the one or more implants.

7. The suturing device of claim 4, wherein an avoidance structure for avoiding the at least two limiting structures is provided on a first implant to be triggered, such that a friction force applied to the first implant to be triggered when the first implant to be triggered passes through the at least two limiting structures is less than a friction force applied to the second implant to be triggered when the second implant to be triggered passes through the at least two limiting structures.

8. The suturing device of claim 4, wherein the puncture needle includes a hollow hole, and a notch communicated with the hollow hole is provided on a circumferential sidewall of the puncture needle; in the axial direction, a front end of the notch is communicated with a tip end surface of the puncture needle, the notch and the hollow hole enclose to form a slot structure, and the slot structure forms the channel;
each of the at least two limiting structures includes a protrusion located on an inner surface of the slot structure; and
two opposite sidewalls of the slot structure are provided with the protrusion, respectively, and the two protrusions are arranged opposite to each other.

9. The suturing device of claim 8, wherein at least a portion of the one or more implants is located in the hollow hole, and when the portion of the one or more implants located in the hollow hole abuts against the notch of the slot structure, a deformation clearance is formed between a bottom surface of the one or more implants and a bottom surface of the slot structure.

10. The suturing device of claim 9, the deformation clearance is within a range of 0.05 mm-0.4 mm.

11. The suturing device of claim 9, wherein each of the one or more implants includes a first portion and a second portion which are connected from top to bottom along a depth direction of the slot structure;
a sidewall of the first portion is provided with a suspension structure, and the first portion is suspended on the notch through the suspension structure, such that a clearance is formed between a bottom of the second portion and an inner sidewall of the hollow hole; and
the second portion is located in the hollow hole, and a width at a widest position of the second portion is greater than a width of the notch.

12. The suturing device of claim 4, further comprising a fixing portion and a triggering mechanism, wherein the puncture needle is connected with the fixing portion, the triggering mechanism is capable of moving along an axial direction relative to the fixing portion, and the triggering mechanism is configured to trigger the one or more implants toward a front end.

13. The suturing device of claim 12, wherein the triggering mechanism includes a push pin, a push button, a connection arm, and a linkage portion; the push button, the connection arm, and the linkage portion are arranged in sequence in a direction from a front end to a rear end of the suturing device;
a front end of the push pin is located in the hollow hole of the puncture needle; and
the push button is slidably connected with the fixing portion; a front end of the connection arm is connected with the push button, and a rear end of the connection arm is connected with the linkage portion; the linkage portion is connected with a rear end of the push pin.

14. The suturing device of claim 13, wherein a spring is provided between the fixing portion and the linkage portion.

15. The suturing device of claim 8, further comprising a depth limiting tube and a locking mechanism, wherein the depth limiting tube sleeves an outer side of the puncture needle, a front end of the depth limiting tube is a depth limiting end; a position of the depth limiting tube in the axial direction is adjustable; and
the locking mechanism is configured to lock the depth limiting tube such that the position of the depth limiting tube is fixed relative to the puncture needle.

16. The suturing device of claim 15, wherein the locking mechanism includes a control arm, the control arm includes a first connection arm, a pressing arm, and a second connection arm which are connected in sequence along the axial direction, one end of the first connection arm is connected with the depth limiting tube, and the other end of the first connection arm is connected with one end of the pressing arm; one end of the second connection arm is connected with the other end of the pressing arm, and the other end of the second connection arm is slidably connected with the fixing portion;
a clearance is provided between the pressing arm and the fixing portion such that the pressing arm is capable of moving relative to the fixing portion; a gear pin is provided on the pressing arm;
the locking mechanism further includes a plurality of limiting slots located on a housing, and the plurality of limiting slots are arranged in sequence along the axial direction; and
the pressing arm has a locked state and an unlocked state, in the locked state, the gear pin is located in one of the plurality of limiting slots, and the limiting slot is configured to prevent the pressing arm from moving along the axial direction, and in the unlocked state, the gear pin is pressed out of the limiting slot, and the pressing arm is capable of moving along the axial direction.

17. The suturing device of claim 16, wherein a first guide slot extending along the axial direction is provided on the fixing portion, a sliding abutting portion is provided on the first connection arm, the sliding abutting portion abuts against the first guide slot, and the first guide slot is configured to guide the first connection arm to slide along the axial direction of the puncture needle.

18. The suturing device of claim 4, further comprising a housing and a triggering mechanism, wherein the triggering mechanism is configured to trigger the one or more implants toward a front end; and
a gear slot is disposed on an inner wall of the housing; an elastic swing rod is disposed on the triggering mechanism, a fixed end of the elastic swing rod is fixedly connected with the triggering mechanism, and a free end of the elastic swing rod extends into the gear slot from the notch of the gear slot.

19. The suturing device of claim 18, wherein a count of implants disposed in the suturing device are two, and the gear slot includes a first slot unit and a second slot unit;
the gear slot further includes a first retreat slot, a bottom surface of the first retreat slot is lower than a bottom surface of the first slot unit; one end of the first retreat slot is located on a bottom surface of a first limiting end of the first slot unit, and extends from the first limiting end toward a rear end and passes through a sidewall of the first slot unit to communicate with the second slot unit, and an angle is formed between an extension direction of the first retreat slot and an extension direction of the first slot unit; a bottom surface of the second slot unit is lower than the bottom surface of the first retreat slot;
the gear slot further includes a second retreat slot, a bottom surface of the second retreat slot is lower than the bottom surface of the second slot unit; one end of the second retreat slot is located on a bottom surface of a second limiting end of the second slot unit, and extends from the second limiting end toward the rear end and communicates with a first starting end of the first slot unit; and
in an initial state, the free end is located at the first starting end, and the free end has a tendency to move toward the bottom surface of the gear slot, such that the free end always abuts against the bottom surface of the gear slot.

20. The suturing device of claim 18, wherein a count of implants disposed in the suturing device are two, and the gear slot includes the first slot unit and the second slot unit;
the first slot unit includes a first slot wall and a second slot wall, an extension direction of the first slot wall is parallel to the axial direction; and a width between the second slot wall and the first slot wall gradually decreases in a direction from the rear end to the front end;
the second slot unit is located on a side where the second slot wall of the first slot unit is located;
the first retreat slot is provided between the first slot unit and the second slot unit, a front end of the first retreat slot is connected with the first limiting end of the first slot unit through a first transverse slot, and a rear end of the first retreat slot is connected with a second starting end of the second slot unit through a second transverse slot;
the second retreat slot is provided on a side of the second slot unit, and the second slot unit is located between the first retreat slot and the second retreat slot; a front end of the second retreat slot is connected with the second limiting end of the second slot unit through a third transverse slot, and a rear end of the second retreat slot is connected with the second slot wall, and a bottom surface of the rear end of the second retreat slot is higher than a bottom surface of the first starting end of the first slot unit, such that a lower step structure is formed at a connection between the second retreat slot and the first slot unit;
in the initial state, the free end is located in the first slot unit and behind an opening of the rear end of the second retreat slot, and abuts against the second slot wall.
